(19) United States
(12) Patent Application Publication
(10) Pub. No.: US 2017/0157284 A1
(43) Pub. Date: Jun. 8, 2017

(54) SILICONE ADHESIVES TO SECURE MEDICAL APPLIANCES TO MAMMALIAN BODY

(71) Applicant: TRIO HEALTHCARE LTD, Skipton, North Yorkshire (GB)

(72) Inventors: Lloyd Pearce, Skipton (GB); John Chacksfield, Skipton (GB); Mahesh Sambasivam, Pennington, NJ (US)

(21) Appl. No.: 15/118,437

(22) PCT Filed: Feb. 9, 2015
(86) PCT No.: PCT/GB2015/050344

§ 371 Date: Aug. 11, 2016

Related U.S. Application Data
(60) Provisional application No. 61/938,401, filed on Feb. 11, 2014.

(30) Foreign Application Priority Data
Mar. 27, 2014 (GB) 1405483.7

(51) Int. Cl. A61L 24/04; A61L 15/26; A61L 24/00; A61L 15/18; A61F 5/449; C09J 11/04; C08K 3/36; C09J 5/06; C09J 7/02; A61L 15/58; C09J 183/04

(52) U.S. Cl. CPC A61L 24/046; A61L 15/585; A61L 15/26; A61L 24/0094; A61L 24/0089; A61L 15/18; C09J 183/04; C09J 11/04; C08K 3/36; C09J 5/06; C09J 7/0264; A61F 5/449; A61L 2400/14; C09J 2205/102; C09J 2483/00

(57) ABSTRACT

The present disclosure relates to the field of skin friendly silicone adhesive compositions and use of such compositions to secure medical appliances to mammalian body, and to protect and treat peri-skin surface.

### BACKGROUND

Technical Field

The present disclosure relates to the field of skin friendly silicone adhesive compositions and use of such compositions to secure medical appliances to mammalian body.

Background

There are medical conditions such as ostomy, pressure ulcer, fistula, chronic and acute wounds, highly exuding wounds, and fecal incontinence that require management of bodily fluids and waste. The management of such fluids and waste is critical in improving the condition such as related to wound healing, and maintaining a quality of life in the case of ostomy and fecal incontinence. Devices or appliances used to manage the above conditions are secured to the body using skin adhesives. Skin adhesives are also used to secure intra-venous or IV fluid lines, and insulin pumps to the body.

In the case of ostomy, the collection bag and adhesive wafer, either as separate components (referred to as “2-piece system”) or permanently jointed together (referred to as “1-piece system”) is attached to the peristomal skin through the adhesive wafer to manage stomal waste. It is challenging to securely attach an ostomy device or appliance to an abdominal stoma due to anatomical contour, skin folds or creases, irregular-shaped stomas, surgical scars, etc. While the adhesion of the adhesive has to be securely maintained while the device is in use, at the time of device change or removal, the adhesive should remove from the skin without causing trauma. This balance in secure adhesion while providing non-traumatic removal is very critical to the successful management of the medical condition. In order to protect the peristomal skin from stomal effluent, ostomates use adhesive discs such as cohesive seal or moldable ring, which form a dam or gasket around the peristomal skin. In some cases, these are convex-shaped to fit the profile of the peristomal contour of the abdomen. These adhesive discs are stretched to fit around the stoma, and pressed down to adhere to the skin. The ostomy wafer or bag is then placed on top of this adhesive gasket. The key properties for a useful adhesive disc or seal or ring for this purpose are its ability to stretch (or low elasticity) and maintain the shape, high tack and adhesion to skin, and good adhesion to the ostomy appliance.

In the case of wound care, dressings are used to manage the exudate and to promote wound healing. Wounds can occur in any part of the body, and depending on the location, it could be challenging to adhere a dressing to the wound. Similar situation arises in fistula, perianal skin management, fecal incontinence, where the anatomy of the body renders it difficult to securely adhere or attach devices to manage the exudate. In negative pressure wound therapy (NPWT) systems used for highly exuding wounds, a vacuum suction is applied to the dressing to displace the exudate from the wound bed and dressing. The securement of such dressings to the peri-wound area is critical to achieve the negative pressure gradient. An adhesive disc could be used to improve the securement of such devices around the wound or fistula.

There are several commercially available pressure sensitive adhesives (PSA) used as skin adhesives which are based on styrenic block copolymers, polyisobutylene, polyethylene, poly(ethylene-vinyl acetate) (EVA), acrylics, and polyurethane chemistries. PSAs are generally more viscoelastic than elastic. The balance of the elastic-viscoelastic properties renders them to be useful as skin adhesives and to secure devices to the body.

Most ostomy appliances are secured to the body using pressure sensitive adhesives loaded with absorbent fillers such as hydrocolloids or superabsorbents to manage the moisture and fluids that the adhesives come in contact with during the use of the appliances.

The use of silicone gel adhesives in ostomy is not common due their high elasticity and low tear strength. However, these gel adhesives have been used to secure wound dressings. The elastic nature of the gel adhesives allows them to retain their shape when stretched below their break point and allowed to relax. Also, the adhesion of these gel adhesives under stress is poor, which means when they are stretched to shape and bonded to skin, they relax back to their original shape resulting in delamination from skin. Silicone pressures sensitive adhesives, which are more viscoelastic, have been primarily used in transdermal drug delivery devices. Other chemistries, such as acrylic adhesives, have been widely used in intravenous (IV) tubing securement tapes and also in securing insulin pumps to the body. Due to the residual monomer in these compositions, and their aggressive adhesion, there is a preference for chemistries that do not contain residual monomers or do not affect skin health.

The present disclosure teaches compositions based on silicone gel adhesive compositions with a balance of elastic and viscoelastic properties of the adhesive, and with good adhesion to medical devices.

Prior art

U.S. Pat. No. 8,439,884 discloses a silicone elastomer double-sided tape to form a layer between an ostomy appliance and skin. The patent is silent about the adhesion properties of the adhesive to an ostomy appliance, which is critical to maintaining a liquid-tight seal, and the skin adhesion under moist conditions, which is normal in a per-stomal environment.

U.S. Pat. No. 7,842,752 discloses an skin adhesive composition based on a blend of silicone pressure sensitive adhesive (PSA), a silicone gel, and water absorbing fillers such as alginates, acrylates, cellulose, chitosan, etc.

U.S. Pat. No. 8,124,675 discloses a method to increase the MVTR of a silicone adhesive by the addition of sodium chloride.

U.S. Pat. No. 8,545,468 discloses a component comprising a silicone elastomer to protect the skin around a stoma in combination with a stoma appliance. The patent specifies the dry adhesion to skin but no mention is made of adhesion to skin under moist condition, and the adhesion of the component to the stoma appliance.

WO 2012/003028 A1 discloses the use of organic polyhydroxy compounds that do not affect the cure in silicone gel.

It is the objective of the present disclosure to provide a skin adhesive composition that can protect the skin, secure a device or appliance to the skin, and also maintain, and promote skin health.

### SUMMARY

One objective of this disclosure is to provide a silicone adhesive composition to protect a region of a skin surface or peri-skin of a mammalian body.

Another objective is to secure medical appliances or devices to a peri-skin surface. Such devices include but not limited to catheter, intravenous feeding lines, securement devices, wound dressings, vac therapy devices, ostomy appliances, and the like.

The above objectives are wholly or partially met by devices, articles, appliances, intermediates, compounds, and methods according to the appended claims. Features and aspects are set forth in the appended claims, and in the following description in accordance with the present disclosure.

Accordingly, in one of the aspects, the present disclosure provides a method of forming a cured silicone adhesive composition to protect a region of a skin surface or peri-skin of a mammalian body, comprising the steps of a) mixing adhesive components comprising: 50-90 wt % of uncured silicone gel adhesive comprising a blend of a polydiorganosiloxane with at least one aliphatically unsaturated group, an organosiloxane with at least one silicone-hydride group, and an addition curing catalyst; 1-50 wt % of a non-silicone hydrophilic liquid additive; 0.1-10 wt % of a cohesive strengthening agent; b) coating the above adhesive mixture on a surface; and c) curing the coated adhesive mixture to form the cured adhesive composition on the surface. The surface could be a low surface energy surface, such as a fluorinated release coated liner, or a substrate to which the silicone can permanently bond and anchor to, such as a polymeric substrate. The cured silicone adhesive composition according to the present disclosure has a peel adhesion of 0.20-3.9 N/cm (0.5-10 N/in), 0.39-3.15 N/cm (1-8 N/in), 0.79-2.36 N/cm (2-6 N/in), 1.2-2.0 N/cm (3-5 N/in) or the like. The adhesive composition has a tack of 50-2000 grams, 100-1500 grams, 200-1000 grams, 300-500 grams, or the like.

In aspects, the hydrophilic liquid additive of the present disclosure is included in the range 1%-50%, 5-40%, 10-30%, or 15-20% by weight of the total adhesive composition. The hydrophilic liquid additive of the present disclosure reduces the elasticity of the cured silicone gel adhesive and increases the adhesion of the adhesive. The amount and type of hydrophilic liquid additive is typically chosen based on level of elastic-viscoelastic balance, tack and adhesion required of the cured adhesive. The hydrophilic liquid additive according to the present disclosure comprises any one or a combination of the group comprising: hydroxy acids, polyethylene glycol, polyethylene glycol-polypropylene glycol copolymers, glycerol ethoxylate, triacetin, hyaluronic acid and its derivatives, sodium hyaluronate, propylene glycol, polyglycerol, glycerol and its esters, sodium pyroglumatic acid, caprylyl glycol, propylene glycol, butylene glycol, sorbitol, algae extract, aloe vera, and glyceryl phosphate.

In aspects, the cohesive strengthening agent of the present disclosure comprises any one or a combination of the group comprising: fumed silica, fumed alumina, colloidal silica, nanoclays, silicates, silane treated organic polymers, polymeric metal oxides, non-polymeric metal oxides, and the like. The cohesive strengthening agent improves the tear strength and cohesive strength of the cured adhesive composition of the present disclosure. This agent is typically a particulate filler, which could also increase the viscosity of the uncured adhesive composition. The amount of cohesive agent is selected based on the improvement in strength required of the cured adhesive, and the viscosity levels manageable for processing the liquid uncured adhesive composition of the present disclosure. The cohesive strengthening agent is included in the range 0.1-10 wt %, 0.5-5%, 1.0-4%, or 1.5-3% by weight of the total adhesive composition. The preferred cohesive strengthening agent comprises fumed silica.

In aspects, the uncured silicone gel adhesive has a viscosity less than 150,000 mPa·s (or cP), less than 100,000 mPa·s (or cP), less than 10,000 mPa·s (or cP), or less than 2000 mPa·s (or cP).

In aspects, the silicone gel includes at least one polyorganosiloxane with at least one hydrophilic group selected from hydroxyl, sulfonyl, amino, acrylamido, amido, carboxylic acid or its salts, glyceryl, oxyethylene, and combinations thereof, in addition to the aliphatically unsaturated groups.

Optionally, the composition comprises trace-20 wt % of at least one siloxane resin. The siloxane resin may include at least one MQ resin. The MQ resin has at least one reactive group such as hydroxyl, alkoxy, hydride, or vinyl functionalities.

In other aspects, the present disclosure includes the use of a silicone adhesive to protect peri-anal, peri-stomal, peri-wound, surgical wound, or peri-fistula skin.

In another aspect, an ostomy adhesive to protect a region of a skin surface around a stoma is disclosed, which is formed by curing a mixture comprising: 50-90 wt % of uncured silicone gel adhesive comprising a blend of polydiorganosiloxane with at least one aliphatically unsaturated group, an organosiloxane with at least one silicone-hydride group, and an addition curing catalyst; 1-50 wt % of a non-silicone hydrophilic liquid additive; 0.1-10 wt % of a cohesive strengthening agent; and trace-20 wt % of at least one siloxane resin.

In another aspect, an ostomy adhesive seal comprising an ostomy adhesive is disclosed, which is formed by curing a mixture comprising: 50-90 wt % of uncured silicone gel adhesive comprising a blend of polydiorganosiloxane with at least one aliphatically unsaturated group, an organosiloxane with at least one silicone-hydride group, and an addition curing catalyst; 1-50 wt % of a non-silicone hydrophilic liquid additive; 0.1-10 wt % of a cohesive strengthening agent; wherein the seal has a top surface to adhere to an ostomy appliance, a bottom surface to adhere to a mammalian skin, and a through hole to fit around a stoma. The ostomy adhesive seal has a top surface to adhere to an ostomy appliance, a bottom surface to adhere to a mammalian skin, and a through hole to fit around the stoma. This comes in a pre-formed shape, which can be re-shaped to fit around a stoma. The pre-formed shape could be a disc, a rectangle, an oval, or the like. The through hole could also be a circle, an oval, or any other shape that matches a stoma opening. The ostomy adhesive seal wherein the bond between the ostomy appliance and the ostomy adhesive seal has a peel strength greater than 19.7 g/cm (50 g/in), or greater than 39.4 g/cm (100 g/in), or greater than 59.1 g/cm (150 g/in), or greater than 78.7 g/cm (200 g/in). In addition, the ostomy adhesive seal maintains the bond to peri-skin for greater than 8 hours, greater than 24 hours, greater than 48 hours, or greater than 72 hours.

In another aspect, an ostomy flange extender to secure an ostomy appliance to skin, comprising a substrate and an adhesive is disclosed, wherein the adhesive is formed by curing a mixture comprising: 50-90 wt % of uncured silicone gel adhesive comprising a blend of polydiorganosiloxane with at least one aliphatically unsaturated group, an organosiloxane with at least one silicone-hydride group, and an addition curing catalyst; 1-50 wt % of a non-silicone hydrophilic liquid additive; 0.1-10 wt % of a cohesive strengthening agent; wherein a surface of the cured adhesive is protected by a releasable liner. The substrate disclosed in the ostomy flange extender is a polymeric film selected from: polyolefins, polyvinyls, polyurethanes and polyurethane-ureas, polyvinyl chloride derivatives, polyacrylic and polyacrylates derivatives, polyacrylonitrile, polyesters, cellulosic films, polyimides, polyamides, polyether block amides, epoxy and phenolic plastics, polycarbonates, epoxy resins, fluorinated polymers, polyoxymethylenes, polyphenylene oxides, polysulfones, polyphenyl sulfide, silicones, or polysaccharide based materials.

In another aspect, an ostomy appliance comprising an ostomy adhesive wafer and a collection bag is disclosed, wherein the adhesive wafer comprises a substrate, and an adhesive, wherein the adhesive is formed by curing a mixture comprising: 50-90 wt % of uncured silicone gel adhesive comprising a blend of polydiorganosiloxane with at least one aliphatically unsaturated group, an organosiloxane with at least one silicone-hydride group, and an addition curing catalyst; 1-50 wt % of a non-silicone hydrophilic liquid additive; 0.1-10 wt % of a cohesive strengthening agent; wherein the wafer has a through hole to receive a stoma. The substrate is a polymeric film selected from: polyolefins, polyvinyls, polyurethanes and polyurethane-ureas, polyvinyl chloride derivatives, polyacrylic and polyacrylates derivatives, polyacrylonitrile, polyesters, cellulosic films, polyimides, polyamides, polyether block amides, epoxy and phenolic plastics, polycarbonates, epoxy resins, fluorinated polymers, polyoxymethylenes, polyphenylene oxides, polysulfones, polyphenyl sulfide, silicones, or a polysaccharide based materials.

Furthermore, in another aspect, an adhesive wound dressing to protect a region of skin surface around the wound is disclosed, comprising a fluid absorbing layer and an adhesive to secure the dressing to the skin surface, wherein the adhesive comprises: 50-90 wt % of uncured silicone gel adhesive comprising a blend of polydiorganosiloxane with at least one aliphatically unsaturated group, an organosiloxane with at least one silicone-hydride group, and an addition curing catalyst; 1-50 wt % of a non-silicone hydrophilic liquid additive; 0.1-10 wt % of a cohesive strengthening agent.

### DETAILED DESCRIPTION

100301 Particular embodiments of the present disclosure are described herein below; however, the disclosed embodiments are merely examples of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The cured silicone adhesive composition according to the present disclosure protects a region of a skin surface or peri-skin of a mammalian body and is able to secure appliances or devices to the body. The cured adhesive composition has a balance of elastic-viscoelastic properties, such that it can be shaped to size or fit around a stoma, wound, or fistula, and maintains the fit. The method of forming the cured composition comprises blending the uncured reactive components of silicone gel, a non-silicone hydrophilic liquid additive, a cohesive strengthening agent, and a silicone resin. The adhesive compositions according to the present disclosure provide a balance of elastic and viscoelastic behavior. This behavior can be evaluated by stretching the composition to a certain length below the tearing or breaking point, bonding the adhesive to a surface such as skin and observing the recovery to its original shape. The compositions of the present disclosure do not recover fully to their original length.

Silicone gel adhesives, sometimes referred to as tacky gels, have low peel adhesion, especially when under tension, which occurs, when a mass is stretched and bonded to skin. Also, the adhesion of such gel adhesives to skin, during perspiration or other sources of moisture, could be highly compromised. The addition of the hydrophilic liquid additive according to the present disclosure modifies the elasticity of the gel without compromising the adhesion and tack significantly. To further control the tack and adhesion, and the tear strength of the adhesive composition, a cohesive strengthening agent could be added. Another advantage of the present adhesive composition is the absence of residue on removal of the adhesive from skin surface. This is important for ostomy and wound applications.

In aspects, the silicone gel adhesive of the present disclosure can be cured by reacting at least one polyorganosiloxane with at least one aliphatically unsaturated group with at least one organosiloxane with at least one silicone-hydride (SiH) group in the presence of an addition curing catalyst. The preferred silicone gel adhesives are obtained by reacting an alkenyl-substituted polydiorganosiloxane, preferably a polydimethylsiloxane having silicon-bonded vinyl, allyl or hexenyl groups, and an organosiloxane containing silicon-bonded hydrogen atom and a catalyst for the reaction of the SiH groups with the Si-alkenyl (SiVi) groups, such as a platinum metal or its compounds or its complexes thereof. The ratio of SiVi:SiH can be 10:1 to 1:10. Preferred ratio of SiVi:SiH is 1:1. Altering the ratio of the reacting silicones from 1:1 ratio can change the adhesive properties of the gel. If a firmer, lower tack gel is required, the SiH component is higher than SiVi, and if a softer gel with higher tack is required, the SiVi component is higher than SiH. The silicone gel compositions can be cured at normal ambient temperatures, but curing times can be reduced by exposure to elevated temperatures, from about 40° C. to about 150° C. Non-limiting examples of such silicone gel adhesives are Soft Skin Adhesives SSA 7-9900, 7-9950 from Dow Corning Corporation, SILPURAN® 2130, SilGel® 612 from Wacker Chemicals. Hydrophilic group containing silicones, according to the present disclosure, may contain polar groups such as acid, amido, amino, sulfonyl, carboxyl, phosphate, phosphonate, etc., on the polydimethylsiloxane backbone. These groups could be present in an ionic form.

In aspects, the adhesive compositions according to the present disclosure comprise at least one liquid hydrophilic additive. The liquid additive of the present disclosure has some miscibility to the silicone gel fluids such than a stable emulsion is formed on complete mixing. Such miscible hydrophilic liquids have been found to not phase separate to the cured surface, which could result in a poor adhesive. The liquid hydrophilic additive can be incorporated into the silicone gel following conventional techniques of blending or mixing additives into silicone gels. For example, the hydrophilic liquid additive could be blended into either Part A or Part B of the silicone gel composition as a pre-mix, or at the meter mixer when the two parts of the gel are mixed together prior to curing the adhesive. It is desirable to obtain a smooth mixture of the adhesive components, either as a suspension or emulsion, such that the mixture is stable over a few minutes that could be required to processing the adhesive for curing reaction. The adhesive mixtures according to the present disclosure provide smooth mixtures suitable for coating, printing, or other processing techniques. In order to adjust the tack and adhesion level of the adhesive composition, the ratio of the two parts of the silicone gel may also be altered from the recommended ratio from the gel manufacturer.

The non-silicone hydrophilic liquid additive according to the present disclosure may at least be partially soluble or miscible in water. The viscosity of the hydrophilic liquid additive could be less than 100,000 mPa·s (or cP), less than 50,000 mPa·s (or cP), preferably less than 10,000 mPa·s (or cP), most preferably less than 1000 mPa·s (or cP). In cases where the liquid additive may retain moisture during storage, the additive could be dried in a desiccator, or in an oven, or other known drying methods prior to addition to the silicone gel. Non-limiting examples of such non-silicone liquid additives are hydroxy acids, polyethylene glycol, polyethylene glycol-polypropylene glycol copolymers, glycerol ethoxylate, triacetin, hyaluronic acid and its derivatives, sodium hyaluronate, propylene glycol, polyglycerol, glycerol and its esters, sodium pyroglumatic acid, caprylyl glycol, propylene glycol, butylene glycol, sorbitol, algae extract, aloe vera, glyceryl phosphate, and combinations thereof. Hydrophilic liquid additives according to the present disclosure yield cured silicone adhesive compositions that have good adhesion to mammalian skin.

In aspects, the adhesive in accordance with the present disclosure includes at least one cohesive strengthening agent. These agents improve the cohesive strength of the gel without compromising the adhesive properties significantly. Cohesive strengthening agents are known to reinforce the tensile and tear strength of silicone rubber. However, not all cohesive strengthening agents yield the same effect when an adhesive gel is required. The agents according to the present disclosure disperse well in the uncured adhesive matrix. The particle size of such agents according to the present disclosure is less than 100 microns, less than 50 microns, preferably less than 10 microns, most preferably, less than 1 micron. Non-limiting examples of cohesive strengthening agents of the present disclosure are silica, which could be fumed or precipitated silica such as AEROSIL® and SIPERNAT® grades, respectively, from Evonik Industries. The silica powders could be hydrophilic or hydrophobic, such as AEROSIL® 300, AEROSIL® 255, AEROSIL® R 812, AEROSIL® R 812 S, SIPERNAT® 120, SIPERNAT® 218, etc. Other non-limiting examples of cohesive strengthening agents include fumed alumina, colloidal silica, nanoclays, silicates, silane treated organic polymers, polymeric metal oxides, non-polymeric metal oxides, and the like. Since the cohesive strengthening agents are typically in particulate form with high surface area, dispersing the agents into the liquid silicone may require high shear mixing to ensure complete mixing and to break down agglomerates of the agent.

In aspects, the adhesive in accordance with the present disclosure may include at least one siloxane resin. Silicone resins are known to increase the adhesion of a silicone adhesive to skin or any substrate. They are also referred to as tackifiers for silicones. Silicone resins are silicone materials formed by branched, cage-like oligosiloxanes with the general formula of RnSiXmOy, where R is a non reactive substituent, usually Me or Ph, and X is a functional group H, OH, vinyl, or OR. These groups are further condensed in many applications, to give highly crosslinked, polysiloxane networks. Typical siloxane resins are MQ resins. MQ resins are three-dimensional network of M type and Q type silicon-oxygen structure. Non-limiting examples of commercially available MQ resins are MQ-RESIN POWDER 803 TF from Wacker Chemical Corporation; VQM-135, VQM-146, HQM-105, HQM-107, SQO-299, and SQD-255 from Gelest Inc., Prosil 9932, MQOH-7 from SiVance, LLC. The resins could have specific functionality such as hydroxyl, vinyl, hydride, and the like. Depending on the resin type and the molecular weight, they are either sold as powders or flakes, or as a solution in a solvent. The resins can be blended into the silicone gel Part A or Part B depending on the resin's functionality, prior to blending both parts together prior to curing reaction. Silicone resins are very expensive, and they tend to increase the tack significantly at the expense of peel adhesion. The resin amount in the adhesive composition of the present disclosure is from trace-20%, 0.5-15%, preferably 1-10%, most preferably 2-8% of the total adhesive composition.

The method of forming the cured silicone adhesive composition according to the present disclosure includes manufacturing processes such as web coating, printing, molding, etc. Those skilled in the art can appreciate that the catalyst used in addition cured silicones are very sensitive, and caution should taken to avoid any poisoning of the catalyst. Typically, the hydrophilic ingredients could be added to the non-catalyst part of the liquid reactive silicone in a two-part system. For one-part RTVs, this could require processing the composition immediately after adding all the ingredients. When curing the adhesive of the present disclosure on a substrate, primers, adhesion promoters, or other surface treatment methods could be employed to improve the adhesion of the adhesive to the substrate. When curing the adhesive of the present disclosure on a releasable liner, a suitable time and temperature condition besides the appropriate liner material has to be chosen. Such liner materials will not result in lock-up of the adhesive and can be removed from the liner for use.

### EXAMPLES

Table 1 shows the examples according to the present disclosure. The silicone gel, Parts A and B, are weighed out in a plastic cup. The hydrophilic liquid additive and the other additives are then added to the silicone gel in the cup. The contents of the cup are mixed together by stirring with a stainless steel spatula. After thoroughly mixing the composition, a uniform coating is applied to a fluorinated release liner or a polyurethane (PU) film using a bird applicator from Byk-Chemie. The adhesive coated on release liner or PU film is cured in a convection oven at 130 C for 30 minutes. The cured adhesive surface is then protected with another fluorinated release liner after cooling to room temperature. For cohesive seal examples, the adhesive mixture is poured onto the fluorinated release liner and cured to form a disc of about 2 mm. The stretchability and elasticity of the composition is evaluated by gently stretching the cooled adhesive disc by hand.

Ingredients List:

MG7-9900—Dow Corning Corporation; SILPURAN® 2130, SilGel® 612—Wacker Chemical Corporation; MED-6340 and MED-6342—Nusil Inc.; Prosil 9932 resin solution—SiVance, LLC; MQOH-7 MQ resin—SiVance, LLC Glycerol—Sigma-Aldrich Chemical; Glycerol ethoxylate 441864 (MW ˜1000 g/mol)—Sigma Aldrich Chemical; Hyaluronic acid—Timeless Skincare; AEROSIL® 300 Pharma—Evonik Industries; Fumed silica 55505-100 g—Sigma-Aldrich Chemical.

TABLE 1
Examples

| | Composition [wt %] | | | | | | | | | |
|---|---|---|---|---|---|---|---|---|---|---|
| | Con- | | | Con- | | Con- | | | Con- | |
| Ingredients | trol 1 | 1A | 1B | trol 2 | 2A | trol 3 | 3A | 3B | trol 4 | 4A |
| MG 7-9900 | 100 | 91 | 75 | | | | | | | |
| Part A + B (1:1) | | | | | | | | | | |
| SILPURAN [symbol] 2130 | | | | | | 100 | 91 | 57 | | |
| Part A + B (1:1) | | | | | | | | | | |
| MED-6342 | | | | 100 | 75 | | | | | |
| Part A + B (1:1) | | | | | | | | | | |
| MED-6340 | | | | | | | | | | |
| Part A + B (1:1) | | | | | | | | | | |
| MED-634 [symbol] | | | | | | | | | | |
| Part A + B (1:1) | | | | | | | | | | |
| LR3003/10 | | | | | | | | | | |
| Part A + B (1:1) | | | | | | | | | | |
| SilGel [symbol] 612 | | | | | | | | | 100 | 91 |
| Part A + B (1:1) | | | | | | | | | | |
| Polyethyl- | | | | | | | | | | |
| eneglycol 200 | | | | | | | | | | |
| Glycerol | | 9 | 24 | | 24 | | 9 | 33 | | |
| Glycerol ethoxylate | | | | | | | | | | |
| Hyaluronic acid | | | | | | | | | | 9 |
| Prosil 9932 | | | | | | | | | | |
| resin solution | | | | | | | | | | |
| MQOH-7 resin | | | | | | | | | | |
| AEROSIL [symbol] | | | 0.7 | | 0.7 | | | | | |
| 300 Pharma | | | | | | | | | | |
| Fumed silica | | | | | | | | | | |

Properties

| | | | | | | | | | | |
|---|---|---|---|---|---|---|---|---|---|---|
| Mixed Gel viscosity | 5100 | NM | NM | 8000 | NM | 1000 | NM | NM | 1000 | NM |
| [manufacturer | mPa · s | | | mPa · s | | mPa · s | | | mPa · s | |
| data] cP or | (or cP) | | | (or cP) | | (or cP) | | | (or cP) | |
| m · Pa · s | | | | | | | | | | |
| Pre-cure mixture | Smooth | Smooth | Smooth | Smooth | Smooth | Smooth | Smooth | Smooth | Smooth | Smooth |
| appearance | | | | | | | | | | |
| *Dry adhesion | med | poor | med | low | low | high | very | very | low | med |
| or tack; | adh & | adh & | adh & | adh & | adh & | adh & | high | high | adh & | adh & |
| residue level | high tack; | tack; | high tack; | high tack; | tack; | tack; | adh & | adh & | high tack; | high tack; |
| | no residue | residue | high | no residue | high | no residue | tack; | tack; | no residue | no residue |
| | | | residue | | residue | | low | low | | |
| | | | | | | | residue | residue | | |
| Recovery | 100% | Medium | Medium | 100% | Medium | 100% | Medium | Medium | 100% | Medium |
| on stretching | recovery | recovery; | recovery; | recovery | recovery; | recovery | recovery; | recovery; | recovery | recovery; |
| below tear/ | | breaks | med | | med | | breaks | breaks | | breaks |
| break point | | easily, poor | cohesive | | cohesive | | easily, poor | easily, poor | | easily, poor |
| (~1.5 inch | | cohesive | strength | | strength | | cohesive | cohesive | | cohesive |
| disc cured | | | | | | | | | | |
| on release liner) | | | | | | | | | | |

| | Composition [wt %] | | | | | | | | |
|---|---|---|---|---|---|---|---|---|---|
| | | | | | | | | Con- | |
| Ingredients | 4B | 4C | 4D | 4E | 4F | 4G | 4H | trol 5 | 5A |
| MG 7-9900 | | | | | | | | | |
| Part A + B (1:1) | | | | | | | | | |
| SILPURAN [symbol] 2130 | | | | | | | | | |
| Part A + B (1:1) | | | | | | | | | |
| MED-6342 | | | | | | | | | |
| Part A + B (1:1) | | | | | | | | | |
| MED-6340 | | | | | | | | | |
| Part A + B (1:1) | | | | | | | | | |
| MED-634 [symbol] | | | | | | | | 100 | 91 |
| Part A + B (1:1) | | | | | | | | | |
| LR3003/10 | | | | | | | | | |
| Part A + B (1:1) | | | | | | | | | |
| SilGel [symbol] 612 | 91 | 77 | 75.5 | 71.4 | 67 | 83.2 | 83.2 | | |
| Part A + B (1:1) | | | | | | | | | |
| Polyethyl- | 9 | 29 | 22.6 | 28.6 | 33 | | | | |
| eneglycol 200 | | | | | | | | | |
| Glycerol | | | | | | | | | 9 |
| Glycerol ethoxylate | | | | | | 7.6 | 7.6 | | |
| Hyaluronic acid | | | | | | | | | |
| Prosil 9932 | | | | | | 7.6 | | | |
| resin solution | | | | | | | | | |
| MQOH-7 resin | | | | | | | 7.6 | | |
| AEROSIL [symbol] | | | | | | | | | |
| 300 Pharma | | | | | | | | | |
| Fumed silica | | | 1.9 | | | 1.6 | 1.6 | | |
| Properties | | | | | | | | | |
| Mixed Gel viscosity | NM | NM | NM | NM | NM | NM | NM | 15,300 | NM |
| [manufacturer | | | | | | | | mPa · s | |
| data] cP or | | | | | | | | (or cP) | |
| m · Pa · s | | | | | | | | | |
| Pre-cure mixture | Smooth | Smooth | Smooth | Marginally | Not smooth | Smooth | Smooth | Smooth | Smooth |
| appearance | | | | smooth | snozzy | | | | |
| *Dry adhesion | med | high | high | high | phase | High | High | med | high |
| or tack; | adh & | adh & | adh & | adh & | separated; | adh & | adh & | adh & | adh & |
| residue level | high tack; | tack; | tack; | tack; | low adh & | tack; | tack; | high tack; | tack; |
| | no residue | slight | slight | some phase | tack; | slight | no residue | no residue | no residue |
| | | residue | residue | separation | high | residue | | | |
| | | | | | residue | | | | |
| Recovery | Medium | Medium | Medium | Medium | NM | Medium | High | 100% | Medium |
| on stretching | recovery; | recovery; | recovery; | recovery; | | recovery; | recovery; | recovery | recovery; |
| below tear/ | breaks | breaks | med | breaks | | med | high | | breaks |
| break point | easily, poor | easily, poor | cohesive | easily, poor | | cohesive | cohesive | | easily, poor |
| (~1.5 inch | cohesive | cohesive | strength; | cohesive | | strength | strength | | cohesive |
| disc cured | | | | | | | | | |
| on release liner) | | | | | | | | | |

*Finger test: for dry, finger was pressed onto adhesive surface and withdrawn after few seconds: [symbol]
indicates data missing or illegible when filed

It can be seen from Table 1 that the silicone gel adhesives without any hydrophilic liquid additive, samples marked “Control” 1-5, have acceptable dry adhesive properties. Since they are elastic gels, the recovery is 100%, which is not desirable for an ostomy application. The hydrophilic liquid additive improves adhesion in some silicone gels and affects adhesion in some. In addition, the hydrophilic additive leads to poor cohesive strength, as shown in examples 1A, 3A, 3B, 4A-C, 4E-F, and 5A under the Recovery on stretching section. When a cohesive strengthening agent such as silica is added in combination with the hydrophilic liquid additive, as shown in examples 1B, 2A, and 4D, a suitable cured adhesive composition with good adhesion, tack, cohesive strength and reduced recovery on stretching is obtained. Further, addition of an MQ resin to the silicone gel adhesive composition along with a liquid hydrophilic additive and cohesive strengthening agent, as shown in examples, 4G and 4H, results in a preferred adhesive composition with the right balance of properties.

Wear Testing:

Adhesive compositions shown in Table 2 were made by coating the adhesives at a thickness of 10 mils using a bird applicator on a PU film (Bioflex 130—2 mils thick from Scapa North America) and cured 130 C for 30 mins. About a 1×1.5 inch strip of tape with each adhesive was adhered to the dry abdominal skin of three people including the inventor. The tapes were worn for 24 hours during normal activities. Results are shown in Table 2.

TABLE 2
Adhesive compositions for wear testing

| | Comparative | Comparative | Inventive | Comparative | Comparative | Inventive |
|---|---|---|---|---|---|---|
| Ingredients | Example 6 | Example 7 | Example 8 | Example 9 | Example 10 | Example 11 |
| Nusil | 100% | 90% | 88.6% | 0 | 0 | |
| MED-6345 | | | | | | |
| (Part A + B) | | | | | | |
| SilGel 612 | 0 | 0 | 0 | 100% | 90% | 88.2% |
| (Part A + B) | | | | | | |
| Glycerol | 0 | 10% | 9.8% | 0 | 10% | 9.8% |
| Fumed | 0 | 0 | 1.6% | 0 | 0 | 2.0% |
| Silica | | | | | | |
| Wear test | Severe edge | Severe edge | No edge | Moderate | Moderate | No edge |
| results | lifting | lifting | lifting | edge lifting | edge lifting | lifting |
| | and low | and medium | and high | and low | and medium | and high |
| | adhesion on | adhesion on | adhesion on | adhesion on | adhesion on | adhesion on |
| | removal | removal | removal | removal | removal | removal |

Tape with Comparative Examples 6, 7, 9, and 10 showed moderate to sever edge lifting, while Examples 8 and 11, according to the present disclosure, showed no edge lifting. In addition, the peel strength of Examples of 8 and 11 were greater than Examples 7 and 10, which were greater than Examples 6 and 9. This clearly demonstrates the benefit of the present disclosure over prior art, and the neat gel adhesives. The combination of the liquid hydrophilic additive and fumed silica, provide a balance in tack, peel strength, and cohesive strength of the adhesive.

1. A method of forming a cured silicone adhesive composition to protect a region of a skin surface or peri-skin of a mammalian body, comprising the steps of a) mixing adhesive components comprising:
i. 50-90 wt % of uncured silicone gel adhesive comprising a blend of a polyorganosiloxane with at least one aliphatically unsaturated group, a organosiloxane with at least one silicone-hydride group, and an addition curing catalyst;
ii. 1-50 wt % of a non-silicone hydrophilic liquid additive;
iii. 0.1-10 wt % of a cohesive strengthening agent; and
b) coating the above adhesive mixture on a surface; and
c) curing the coated adhesive mixture to form the cured adhesive composition on the surface.

2. The method as claimed in claim 1 wherein the adhesive composition has a peel adhesion of 0.20-3.9 N/cm (0.5-10 N/in), 0.39-3.15 N/cm (1-8 N/in), 0.79-2.36 N/cm (2-6 N/in), 1.2-2.0 N/cm (3-5 N/in) or the like.

3. The method as claimed in claim 1 wherein the adhesive composition has a tack of 50-2000 grams, 100-1500 grams, 200-1000 grams, 300-500 grams, or the like.

4. The method as claimed in claim 1 wherein the hydrophilic liquid additive is included in the range 1%-50%, 5-40%, 10-30% or 15-20% by weight of the total adhesive composition.

5. The method as claimed in claim 1 wherein the hydrophilic liquid additive comprises any one or a combination of the group comprising: hydroxy acids, polyethylene glycol, polyethylene glycol-polypropylene glycol copolymers, glycerol ethoxylate, triacetin, hyaluronic acid and its derivatives, sodium hyaluronate, propylene glycol, polyglycerol, glycerol and its esters, sodium pyroglumatic acid, caprylyl glycol, propylene glycol, butylene glycol, sorbitol, algae extract, aloe vera, and glyceryl phosphate.

6. The method as claimed in claim 1 wherein the cohesive strengthening agent comprises any one or a combination of the group comprising: fumed silica, fumed alumina, colloidal silica, nanoclays, silicates, silane treated organic polymers, polymeric metal oxides, and non-polymeric metal oxides.

7. The method as claimed in claim 1 wherein the cohesive strengthening agent is included in the range 0.1-10%, 0.5-5%, 1.0-4% or 1.5-3% by weight of the total adhesive composition.

8. The method as claimed in claim 1 wherein the cohesive strengthening agent comprises fumed silica.

9. The method as claimed in claim 1 wherein the uncured silicone gel adhesive has a viscosity less than 150,000 mPa·s (or cP), less than 100,000 mPa·s (or cP), less than 10,000 mPa·s (or cP), or less than 2000 mPa·s (or cP).

10. The method as claimed in claim 1 wherein the silicone gel includes at least one polyorganosiloxane with at least one hydrophilic group selected from hydroxyl, sulfonyl, amino, acrylamido, amido, carboxylic acid or its salts, glyceryl, oxyethylene, and combinations thereof, in addition to the aliphatically unsaturated groups.

11. The method as claimed in claim 1 comprising a siloxane resin, and preferably a MQ resin.

12. Use of a silicone adhesive in accordance with claim 1 to protect peri-anal, peri-stomal, peri-wound, surgical wound, or peri-fistula skin.

13. An ostomy adhesive to protect a region of a skin surface around a stoma, formed by curing a mixture comprising:
i. 50-90 wt % of uncured silicone gel adhesive comprising a blend of a polyorganosiloxane with at least one aliphatically unsaturated group, an organosiloxane with at least one silicone-hydride group, and an addition curing catalyst;
ii. 1-50 wt % of a non-silicone hydrophilic liquid additive;
iii. 0.1-10 wt % of a cohesive strengthening agent; and

14. An ostomy adhesive seal comprising an ostomy adhesive formed by curing a mixture comprising:
i. 50-90 wt % of uncured silicone gel adhesive comprising a blend of a polyorganosiloxane with at least one aliphatically unsaturated group, an organosiloxane with at least one silicone-hydride group, and an addition curing catalyst;
ii. 1-50 wt % of a non-silicone hydrophilic liquid additive;
iii. 0.1-10 wt % of a cohesive strengthening agent; and
wherein the seal has a top surface to adhere to an ostomy appliance, a bottom surface to adhere to a mammalian skin, and a through hole to fit around a stoma.

15. The ostomy adhesive seal according to claim 14 wherein the bond between the ostomy appliance and the ostomy adhesive seal has a peel strength greater than 19.7 g/cm (50 g/in), or greater than 39.4 g/cm (100 g/in), or greater than 59.1 g/cm (150 g/in), or greater than 78.7 g/cm (200 g/in).

16. The ostomy adhesive seal in accordance with claim 15 wherein the adhesive maintains the bond to peri-skin for greater than 8 hours, greater than 24 hours, greater than 48 hours or greater than 72 hours.

17. An ostomy flange extender to secure an ostomy appliance to skin, comprising a substrate and an adhesive, wherein the adhesive is formed by curing a mixture comprising:
i. 50-90 wt % of uncured silicone gel adhesive comprising a blend of a polyorganosiloxane with at least one aliphatically unsaturated group, an organosiloxane with at least one silicone-hydride group, and an addition curing catalyst;
ii. 1-50 wt % of a non-silicone hydrophilic liquid additive;
ii. 0.1-10 wt % of a cohesive strengthening agent; and
wherein a surface of the cured adhesive is protected by a releasable liner.

18. The ostomy flange extender according to claim 17, wherein the substrate is a polymeric film selected from: polyolefins, polyvinyls, polyurethanes and polyurethane-ureas, polyvinyl chloride derivatives, polyacrylic and polyacrylates derivatives, polyacrylonitrile, polyesters, cellulosic films, polyimides, polyamides, polyether block amides, epoxy and phenolic plastics, polycarbonates, epoxy resins, fluorinated polymers, polyoxymethylenes, polyphenylene oxides, polysulfones, polyphenyl sulfide, silicones or polysaccharide based materials.

19. An ostomy appliance comprising an ostomy adhesive wafer and a collection bag, wherein the adhesive wafer comprises a substrate, and an adhesive, wherein the adhesive is formed by curing a mixture comprising:
i. 50-90 wt % of uncured silicone gel adhesive comprising a blend of a polyorganosiloxane with at least one aliphatically unsaturated group, an organosiloxane with at least one silicone-hydride group, and an addition curing catalyst;
ii. 1-50 wt % of a non-silicone hydrophilic liquid additive;
iii. 0.1-10 wt % of a cohesive strengthening agent; and
wherein the wafer has a through hole to receive a stoma.

20. The ostomy appliance according to claim 19 wherein the substrate is a polymeric film selected from: polyolefins, polyvinyls, polyurethanes and polyurethane-ureas, polyvinyl chloride derivatives, polyacrylic and polyacrylates derivatives, polyacrylonitrile, polyesters, cellulosic films, polyimides, polyamides, polyether block amides, epoxy and phenolic plastics, polycarbonates, epoxy resins, fluorinated polymers, polyoxymethylenes, polyphenylene oxides, polysulfones, polyphenyl sulfide, silicones or a polysaccharide based material.

21. An adhesive wound dressing to protect a region of skin surface around a wound, comprising a fluid absorbing layer and an adhesive to secure the dressing to the skin surface wherein the adhesive comprises:
i. 50-90 wt % of uncured silicone gel adhesive comprising a blend of a polyorganosiloxane with at least one aliphatically unsaturated group, an organosiloxane with at least one silicone-hydride group, and an addition curing catalyst;
ii. 1-50 wt % of a non-silicone hydrophilic liquid additive; and
iii. 0.1-10wt % of a cohesive strengthening agent.
